(19) United States
(12) Patent Application Publication
(10) Pub. No.: US 2021/0153915 A1
(43) Pub. Date: May 27, 2021

(54) Implant Apparatus for Spinal Surgery

(71) Applicant: Solco Biomedical Co., Ltd., Pyeongtaek-si (KR)

(72) Inventors: Mi-rim HYEON, Hwaseong-si Gyeonggi-do (KR); Hwi Geun YU, Pyeongtaek-si Gyeonggi-do (KR); Il KIM, Seoul (KR)

(73) Assignee: Solco Biomedical Co., Ltd., Pyeongtaek-si (KR)

(21) Appl. No.: 16/804,673

(22) Filed: Feb. 28, 2020

(30) Foreign Application Priority Data
Nov. 21, 2019 (KR) 10-2019-0150038

(51) Int. Cl. A61B 17/70; A61B 17/86

(52) U.S. Cl. CPC A61B 17/7082; A61B 17/861

(57) ABSTRACT

Provided is an implant apparatus for spinal surgery, in which a shaft is detachably connected to a connector equipped with a grip part, thus making it easy to replace and clean the shaft.

The implant apparatus for spinal surgery includes a shaft having a predetermined length, with a screw for the spinal surgery being fastened to one end of the shaft, a sleeve fixedly surrounding an outer surface of the shaft, a straight handle and a clamping handle fastened to an outer circumference of the sleeve, a shaft locking part coupled to the clamping handle to lock the shaft, and a connector including an assembly member that is detachably coupled to the other end of the shaft and is assembled with a separate grip part while accommodating the other end of the shaft, and a housing connected to the assembly member and having a shaft coupling space therein to fix or release the inserted shaft.

### BACKGROUND OF THE DISCLOSURE

### Field of the Invention

The present disclosure relates to an implant apparatus for spinal surgery configured to fix a screw to the spine.

### Related Art

Generally, the spine is composed of 24 bones (excluding sacral spine), which are connected by joints called disks between the respective bones to support the spine and absorb shocks. Thereby, the spine can help to maintain the posture of the human, form the basis of movement, and play an important role in protecting internal organs.

However, if an abnormal posture is maintained for a long time, the spine suffers from degenerative diseases due to aging or is subjected to external shocks, the disk of the spinal joint may be damaged, thus resulting in spinal disk diseases. These spinal disk diseases compress nerves connected to respective parts of the human body via the spinal joints, thus causing pain.

Therefore, patients having the spinal disk diseases undergo the following procedure: a disk of a damaged region is removed so that a damaged part of the vertebra is not pressed or compressed, an artificial aid (cage) made of hollow metal or plastic materials is filled with bone chips to be inserted into the region from which the disk has been removed, and then a screw is fixedly inserted into the vertebra of each of upper and lower regions of the damaged disk. Thereafter, a rod is connected to the screw to secure a distance between the vertebrae and thereby allow bone fusion to be normally performed.

The spinal surgery may be performed by incising the skin of a damaged spinal region, removing or leaving the damaged disk, fixedly inserting the screw into the vertebra of each of upper and lower portions of the damaged disk, connecting the rod thereto, and fastening a bolt to the screw of the fixed disk.

That is, a conventional implant apparatus used in the spinal surgery includes a shaft, a sleeve surrounding the shaft, a straight handle assembled on the sleeve, and a clamping handle.

The screw fixed to the spine is assembled on one end of the shaft, while a separate grip part is installed on the other end of the shaft.

However, the grip part is attached or fastened to the shaft by welding or by a screw, so that the shaft is integrated with the grip part. Thus, since it is difficult to separate the shaft, it is difficult to clean the shaft.

When the spinal surgery is performed using the implant apparatus, a patient's blood permeates into the implant apparatus, and it is difficult to clean the blood congealing in the implant apparatus, so that the blood may remain in the implant apparatus and thereby another patient may be undesirably infected during his or her surgery.

Furthermore, since it is difficult to separate the shaft from the implant apparatus, the entire apparatus should be replaced when the shaft is worn, and it is uneconomical.

### CITED REFERENCE

### Patent Document

(Patent Document 1) Korean Patent No. 10-1111666 (Title of Invention: Apparatus of spinal surgical operation for minimally invasive surgery, laid open on Aug. 30, 2010)
(Patent Document 2) Korean Patent Publication No. 10-2019-0108269 (Title of Invention: Spinal surgery device, laid open on Sep. 24, 2019)

### SUMMARY OF THE INVENTION

The present disclosure provides an implant apparatus for spinal surgery, in which a shaft is detachably connected to a connector equipped with a grip part, thus making it easy to replace and clean the shaft.

In an aspect, an implant apparatus for spinal surgery may include a shaft having a predetermined length, with a screw for the spinal surgery being fastened to one end of the shaft, a sleeve fixedly surrounding an outer surface of the shaft, a straight handle and a clamping handle fastened to an outer circumference of the sleeve, a shaft locking part coupled to the clamping handle to lock the shaft, and a connector including an assembly member that is detachably coupled to the other end of the shaft and is assembled with a separate grip part while accommodating the other end of the shaft, and a housing connected to the assembly member and having a shaft coupling space therein to fix or release the inserted shaft.

The shaft locking part may be disposed to be movable on the shaft, and the shaft locking part may engage with the clamping handle to allow the shaft to rotate only in one direction.

An assembly groove may be formed on an outer circumference of the assembly member of the connector so that the separate grip part is assembled therewith.

The connector may include a locker that is installed in the housing to be movable through a compression coil spring and locks the shaft inserted into the housing.

The locker may include a locking hole formed to lock or unlock the shaft.

An elongated hole may be formed in a portion adjacent to the locking hole of the locker, so that the locker may be assembled with the housing by inserting a pin into the elongated hole.

The locker may be fastened to the pin to be movable along the elongated hole.

The present disclosure provides an implant apparatus for spinal surgery, in which a shaft is conveniently assembled with or separated from a connector, thus making it easy to replace and clean the shaft, and thereby effectively keeping the entire apparatus clean.

Furthermore, since it is possible to assemble or separate a shaft with or from an apparatus in a one-touch manner, the apparatus can be more conveniently used, and the worn shaft can be conveniently replaced, thus making it convenient to maintain or repair the apparatus.

### BRIEF DESCRIPTION OF THE DRAWINGS

FIG. 1 is a diagram illustrating an implant apparatus for spinal surgery in accordance with an embodiment of the present disclosure.

FIG. 2 is a diagram illustrating the configuration of the implant apparatus for the spinal surgery in accordance with the embodiment of the present disclosure.

FIG. 3 is a diagram illustrating the configuration of a connector in accordance with the embodiment of the present disclosure.

FIG. 4 is a diagram illustrating the internal structure of the implant apparatus for the spinal surgery in accordance with the embodiment of the present disclosure.

FIG. 5 is a diagram illustrating the internal structure of the connector in accordance with the embodiment of the present disclosure.

FIG. 6 is a diagram illustrating a state in which a screw is fixed to a patient's spine using the implant apparatus for the spinal surgery in accordance with the embodiment of the present disclosure.

### DESCRIPTION OF EXEMPLARY EMBODIMENTS

Hereinafter, an implant apparatus for spinal surgery in accordance with an embodiment of the present disclosure will be described with reference to the accompanying drawings. The present disclosure is not limited or restricted by the embodiment. Further, in describing the present disclosure, specific details of known functions or configurations may be omitted to clarify the gist of the present disclosure.

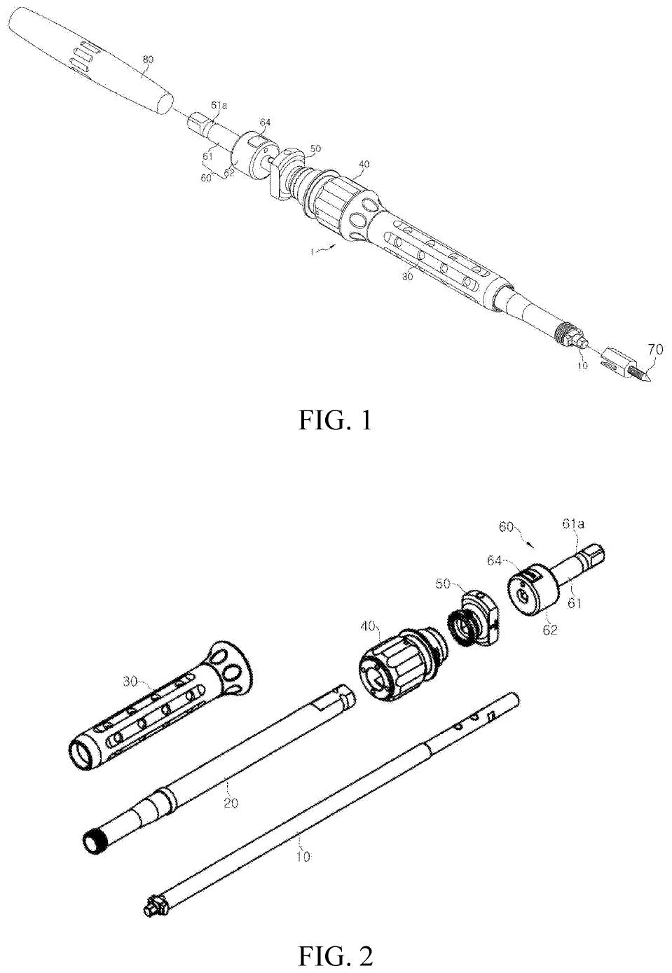

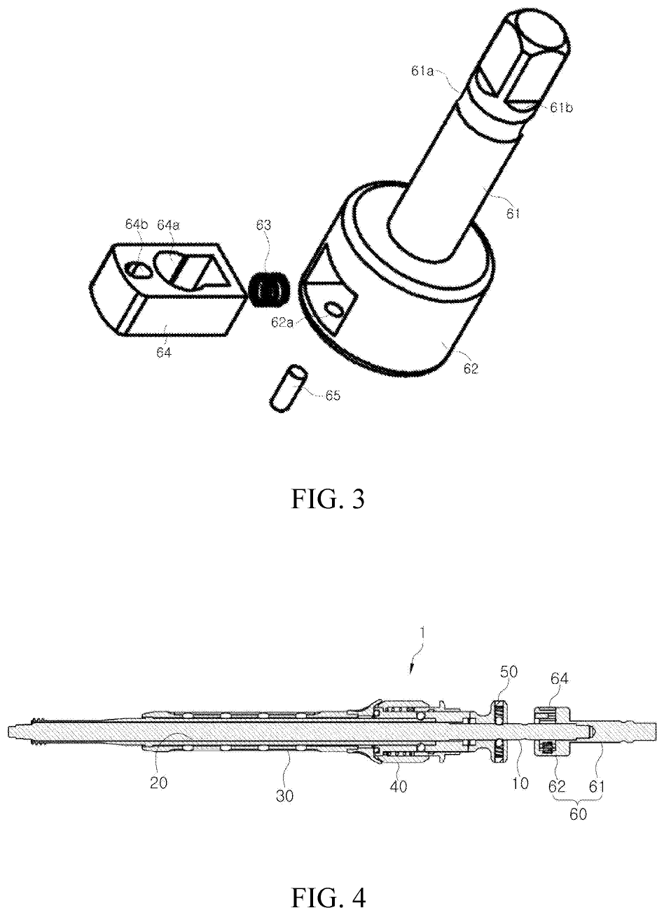

FIG. 1 is a diagram illustrating an implant apparatus for spinal surgery in accordance with an embodiment of the present disclosure. FIG. 2 is a diagram illustrating the configuration of the implant apparatus for the spinal surgery in accordance with the embodiment of the present disclosure. FIG. 3 is a diagram illustrating the configuration of a connector in accordance with the embodiment of the present disclosure.

Referring to FIGS. 1 to 3, the implant apparatus 1 for the spinal surgery in accordance with the embodiment of the present disclosure includes a shaft 10, a sleeve 20, a straight handle 30, a clamping handle 40, a shaft locking part 50, and a connector 60. The implant apparatus 1 may detachably assemble the shaft 10 through the connector 60.

According to this embodiment, the shaft 10 of the implant apparatus 1 has a predetermined length, with a screw 70 for the spinal surgery being fastened to one end of the shaft. The sleeve 20 may fixedly surround an outer surface of the shaft 10, and the straight handle 30 and the clamping handle 40 may be fastened to an outer circumference of the sleeve 20, and the shaft locking part 50 may be coupled to the clamping handle 40 to lock the shaft 10.

Furthermore, the shaft locking part 50 may be disposed to be movable on the shaft 10, and the shaft locking part 50 may engage with the clamping handle 40 to allow the shaft 10 to rotate only in one direction.

If the shaft locking part 50 moves to the clamping handle 40 to be secured to the clamping handle 40 when the implant apparatus 1 is used, the shaft 10 rotates only in one direction and does not run idle in an opposite direction, so that the screw 70 can be more precisely fixed.

In this embodiment, the connector 60 of the implant apparatus 1 may include an assembly member 61 and a housing 62, and may fix or release the other end of the shaft 10 using the connector 60. A separate grip part 80 may be assembled with the connector 60.

That is, the assembly member 61 of the connector 60 may be detachably coupled to the other end of the shaft 10, and may be assembled with the grip part 80 while accommodating the other end of the shaft 10. The housing 62 is connected to the assembly member 61 and has a coupling space for the shaft 10 therein to fix or release the inserted shaft 10.

An assembly groove 61a may be formed on an outer circumference of the assembly member 61 of the connector 60 so that the grip part 80 is assembled therewith. An assembly protrusion 61b may be formed on a portion adjacent to the assembly groove 61a along the outer circumference of the assembly member 61.

The grip part 80 fastened to the connector 60 through the assembly groove 61a and the assembly protrusion 61b formed on the assembly member 61 of the connector 60 can be more firmly fixed.

According to this embodiment, the connector 60 of the implant apparatus 1 may include a locker 64 that is installed in the housing 62 to be movable through a compression coil spring 63 and locks the shaft 10 inserted into the housing 62.

The locker 64 is in elastic contact with the shaft 10 by the compressive force of the compression coil spring 63 to lock the shaft 10. The compression coil spring 63 biases the locker 64 to cause the locker 64 to come into contact with the shaft 10 and thereby lock the shaft 10 to the connector 60.

Furthermore, a locking hole 64a may be formed in the locker 64 to lock or unlock the shaft 10, and an elongated hole 64b may be formed in a portion adjacent to the locking hole 64a of the locker 64, so that the locker 64 may be assembled with the housing 62 by inserting a pin 65 into the elongated hole 64b. When the locker 64 is assembled, the pin 65 may be inserted into a fastening hole 62a formed in a bottom of the housing 62 and simultaneously pass through the elongated hole 64b of the locker 64, thus allowing the locker 64 to be movably assembled with the housing 62.

That is, the locker 64 may be fastened to the pin 65 to be movable along the elongated hole 64b. The shaft 10 may be locked or unlocked by the movement of the locker 64.

Therefore, since the shaft 10 can be more conveniently locked or unlocked using the connector 60, it is possible to more easily maintain and repair the implant apparatus 1.

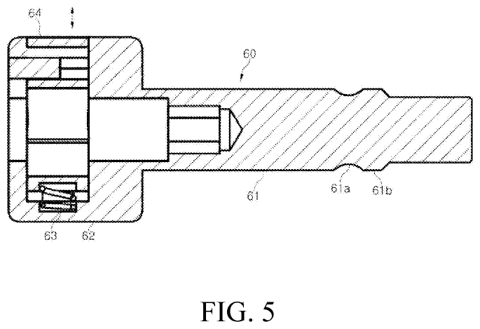

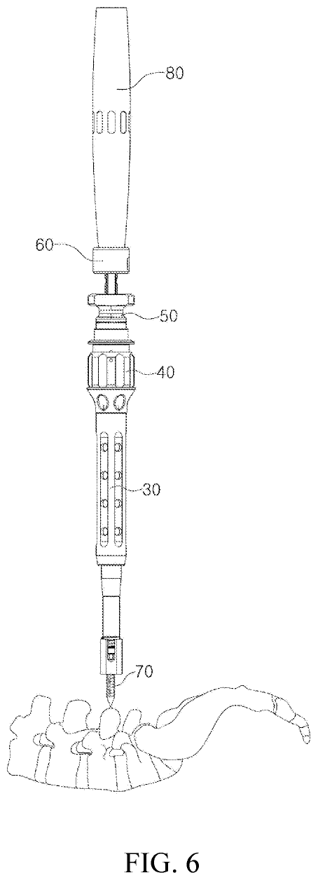

FIG. 4 is a diagram illustrating the internal structure of the implant apparatus for the spinal surgery in accordance with the embodiment of the present disclosure. FIG. 5 is a diagram illustrating the internal structure of the connector in accordance with the embodiment of the present disclosure. FIG. 6 is a diagram illustrating a state in which a screw is fixed to a patient's spine using the implant apparatus for the spinal surgery in accordance with the embodiment of the present disclosure.

Referring to FIGS. 4 to 6, the implant apparatus 1 for the spinal surgery is configured such that the screw 70 is installed at a center of one end of the shaft 10, and the connector 60 is installed on the other end of the shaft 10. Thus, while the connector 60 is fitted over the shaft 10, the connector may be locked by the operation of the locker 64.

That is, in the case of locking the connector 60 to the shaft 10, if a user presses the locker 64 of the connector 60, the locker 64 moves, so that the compression coil spring 63 is compressed. In this state, if the connector 60 is fitted over the shaft 10 and the user releases his or her hand from the locker 64, the locker 64 is moved by the elastic force of the compression coil spring 63 to push and lock the shaft 10.

The shaft 10 inserted into the locking hole 64a of the housing 62 remains fastened to the connector 60 by the movement of the locker 64. The locker 64 remains in contact with the shaft 10 by the force of the compression coil spring 63.

On the other hand, in the case of separating the connector 60 from the shaft 10, if the locker 64 is pushed to release the contact with the shaft 10, the shaft 10 unfastened from the interior of the locking hole 64a may be naturally separated from the connector 60.

The connector 60 may be fitted over or taken out from the shaft 10 in the state where the locker 64 is pushed through the one-touch structure of the locker 64.

Hence, the implant apparatus for fixing the screw to the spine in the spinal surgery may fixedly insert the screw into the spine by rotating the implant apparatus with the user gripping the grip part in the state where the screw is fixed to the shaft.

Therefore, the shaft 10 is conveniently assembled with or separated from the connector 60 through the implant apparatus 1, thus making it easy to replace and clean the shaft 10, and thereby effectively keeping the entire apparatus clean.

Further, since the implant apparatus 1 can be more easily disassembled, it is easy to clean respective components, and the infection of a patient caused by the blood remaining in the implant apparatus 1 can be prevented.

Furthermore, since it is possible to assemble or separate the shaft 10 with or from the apparatus in the one-touch manner, the apparatus can be more conveniently used, and the worn shaft 10 can be conveniently replaced, thus making it convenient to maintain or repair the apparatus.

Although the present disclosure has been shown and described with reference to preferred embodiments for illustrating the principle of the present disclosure, the present disclosure is not limited to the above-described configuration and operation. Rather, those skilled in the art will appreciate that many changes and modifications of the present disclosure may be made without departing from the spirit and scope of the appended claims.

What is claimed is:

1. An implant apparatus for spinal surgery, comprising:
a shaft having a predetermined length, with a screw for the spinal surgery being fastened to one end of the shaft;
a sleeve fixedly surrounding an outer surface of the shaft;
a straight handle and a clamping handle fastened to an outer circumference of the sleeve;
a shaft locking part coupled to the clamping handle to lock the shaft; and
a connector comprising an assembly member that is detachably coupled to the other end of the shaft and is assembled with a separate grip part while accommodating the other end of the shaft, and a housing connected to the assembly member and having a shaft coupling space therein to fix or release the inserted shaft.

2. The implant apparatus of claim 1, wherein the shaft locking part is disposed to be movable on the shaft, and the shaft locking part engages with the clamping handle to allow the shaft to rotate only in one direction.

3. The implant apparatus of claim 1, wherein an assembly groove is formed on an outer circumference of the assembly member of the connector so that the separate grip part is assembled therewith.

4. The implant apparatus of claim 1, wherein the connector comprises a locker that is installed in the housing to be movable through a compression coil spring and locks the shaft inserted into the housing.

5. The implant apparatus of claim 4, wherein the locker comprises a locking hole formed to lock or unlock the shaft.

6. The implant apparatus of claim 5, wherein an elongated hole is formed in a portion adjacent to the locking hole of the locker, so that the locker is assembled with the housing by inserting a pin into the elongated hole.

7. The implant apparatus of claim 6, wherein the locker is fastened to the pin to be movable along the elongated hole.
